(19) United States
(12) Patent Application Publication
(10) Pub. No.: US 2021/0069110 A1
(43) Pub. Date: Mar. 11, 2021

(54) NON-AQUEOUS COMPOSITION HAVING DRUG CARRIED THEREIN, AND METHOD FOR PRODUCING SAME

(71) Applicant: Techno Guard Co. Ltd., Kawasaki-shi (JP)

(72) Inventor: Kiichiro NABETA, Kawasaki-shi (JP)

(73) Assignee: Techno Guard Co. Ltd., Kawasaki-shi (JP)

(21) Appl. No.: 16/499,726

(22) PCT Filed: Mar. 31, 2018
(86) PCT No.: PCT/JP2018/014030

§ 371 Date: Nov. 23, 2020

(30) Foreign Application Priority Data
Mar. 31, 2017 (JP) 2017-070591

(51) Int. Cl. A61K 9/107; A61K 47/44; A61K 47/10

(52) U.S. Cl. CPC A61K 9/107; A61K 31/573; A61K 47/10; A61K 47/44

(57) ABSTRACT

An object of the present invention is to provide a non-aqueous composition having a drug carried therein, the composition being capable of preparing a drug-containing fat emulsion that is usable as an injection, an eye drop, a nasal drop, an inhalant, or the like by being mixed with an aqueous medium in use without producing a drug-containing fat emulsion in advance, and a method for producing the non-aqueous composition. The non-aqueous composition having a drug carried therein of the present invention as a resolution for achieving the object is characterized by comprising an oil or fat, a slightly water soluble drug, and an emulsifier which are dissolved in a polyhydric alcohol as a water soluble carrier at a content of the oil or fat of 0.05 to 250 mg/g, a weight ratio of the slightly water soluble drug to the oil or fat (slightly water soluble drug/(oil or fat)) of 0.0001 to 50 (provided that the total content of the slightly water soluble drug and the oil or fat is at most 300 mg/g), and a content of the emulsifier of 20 to 500 mg/g.

### TECHNICAL FIELD

The present invention relates to a non-aqueous composition having a drug carried therein, the composition being capable of preparing a drug-containing fat emulsion by being mixed with an aqueous medium, such as water for injection and a physiological saline, in use, and a method for producing the non-aqueous composition.

### BACKGROUND ART

It is well known to persons skilled in the art that some drug-containing fat emulsions, such as a steroid (dexamethasone palmitate)-containing fat emulsion and a prostaglandin (PGE₁) -containing fat emulsion, have been already placed on the market and commonly used. However, some of them have some limitations, for example, have poor stability and thus are required to be stored in a cold place.

In a known example of a method for enhancing storage stability of a drug-containing fat emulsion, the aqueous phase is removed from the emulsion o maintain the resultant in a dry state. However, when a heretofore-proposed method in which an emulsion is freeze-dried at a temperature of minus several tens degrees Celsius is used as a method for removing the aqueous phase from the emulsion, a time and cost are required. Accordingly, there is a need for a method for enhancing storage stability of a drug-containing fat emulsion by drying the emulsion under milder conditions to remove the aqueous phase from the emulsion.

Thus, the present inventor proposes in Patent Document 1 a method for producing a non-aqueous composition containing fat particles having a drug carried therein under mild drying conditions, the composition being capable of preparing a drug-containing fat emulsion that is usable as an injection, an eye drop, a nasal drop, an inhalant, or the like by being mixed with an aqueous medium in use.

### PRIOR ART DOCUMENTS

### Patent Document

Patent Document 1: JP-A-2010-270023

### SUMMARY OF THE INVENTION

### Problems that the Invention is to Solve

The non-aqueous composition containing fat particles having a drug carried therein proposed in Patent Document 1 by the present inventor can be produced from a drug-containing fat emulsion that is produced so that the content of an oil or fat is at most 2 mg/mt, and a drug-containing fat emulsion can be prepared by mixing the non-aqueous composition with an aqueous medium in use. However, for producing the non-aqueous composition, a drug-containing fat emulsion has to be produced in advance, and the method is thus time and labor consuming.

Thus, an object of the present invention is to provide a non-aqueous composition having a drug carried therein, the composition being capable of preparing a drug-containing fat emulsion that is usable as an injection, an eye drop, a nasal drop, an inhalant, or the like by being mixed with an aqueous medium in use without producing a drug-containing fat emulsion in advance, and a method for producing the non-aqueous composition.

### Means for Solving the Problems

As a result of intensive and extensive studies in view of the above situation, the present inventor has found that a non-aqueous composition that can prepare a drug-containing fat emulsion by being mixed with an aqueous medium can be obtained by dissolving an oil or fat, a drug, and an emulsifier in a polyhydric alcohol as a water soluble carrier so that the content of the oil or fat, the weight ratio of the drug to the oil or fat, the total content of the drug and the oil or fat, and the content of the emulsifier are within suitable numeral ranges.

A non aqueous composition having a drug carried therein of the present invention made on the basis of the above findings is, as described in claim 1, characterized by comprising an oil or fat, a slightly water soluble drug, and an emulsifier which are dissolved in a polyhydric alcohol as a water soluble carrier at a content of the oil or fat of 0.05 to 250 mg/g, a weight ratio of the slightly water soluble drug to the oil or fat (slightly water soluble drug/(oil or fat)) of 0.0001 to 50 (provided that the total content of the slightly water soluble drug and the oil or fat is at most 300 mg/g), and a content of the emulsifier of 20 to 500 mg/g.

The non-aqueous composition described in claim 2 is characterized, in the non-aqueous composition according to claim 1, in that the polyhydric alcohol is at least one selected from glycerol, propylene glycol, and polyethylene glycol.

A method for producing a non-aqueous composition having a drug carried therein of the present invention is, as described in claim 3, characterized by comprising dissolving an oil or fat, a slightly water soluble drug, and an emulsifier in a polyhydric alcohol as a water soluble carrier at a content of the oil or fat of 0.05 to 2.50 mg/g, a weight ratio of the slightly water soluble drug to the oil or fat (slightly water soluble drug/(oil or fat)) of 0.0001 to 50 (provided that the total content of the slightly water soluble drug and the oil or fat is at most 300 mg/g), and a content of the emulsifier of 20 to 500 mg/g.

A pharmaceutical preparation of the present invention is, as described in claim 4, characterized by comprising the non-aqueous composition having a drug carried therein according to claim 1 alone or together with another component blended therein.

### Effect of the Invention

The present invention can provide a non-aqueous composition having a drug carried therein, the composition being capable of preparing a drug-containing fat emulsion that is usable as an injection, an eye drop, a nasal drop, an inhalant, or the like by being mixed with an aqueous medium in use without producing a drug-containing fat emulsion in advance, and a method for producing the non-aqueous composition.

### Mode for Carrying out the Invention

The non-aqueous composition having a drug carried therein of the present invention is characterized by comprising an oil or fat, a slightly water soluble drug, and an emulsifier which are dissolved in a polyhydric alcohol as a water soluble carrier at a content of the oil or fat of 0.05 to 250 mg/g, a weight ratio of the slightly water soluble drug to the oil or fat (slightly water soluble drug/(oil or fat)) of 0.0001 to 50 (provided that the total content of the slightly water soluble drug and the oil or fat is at most 300 mg/g), and a content of the emulsifier of 20 to 500 mg/g.

In the present invention, the slightly water soluble drugs include slightly water soluble drugs having a solubility, in terms of the solubility in water defined in General Notice of Japanese Pharmacopoeia, of “sparingly soluble” (the volume of solvent required for dissolving 1 g or 1 mL of solute is from 30 mL to less than 100 mL: the solute corresponds to the drug and the solvent corresponds to water) or lower solubility, and more suitably having a solubility of “slightly soluble” (the volume of solvent of the same is from 100 to less than 1000 ml) or lower solubility, and further suitably having a solubility of “very slightly soluble” (the volume of solvent of the same is from 1000 to less than 10000 mL) or lower soluble and most suitably having a solubility of “practically insoluble” (the volume of solvent of the same is 10000 mL or over) or lower solubility. The drug may be slightly oil soluble while being slightly water soluble. The type of the drug is not practically limited, and examples thereof include immunosuppressive drugs, such as cyclosporin and tacrolimus; antibiotics, such as erythromycin and clarithromycin; anti-inflammatory analgesics, such as indometacin, aspirin, ibuprofen, ketoprofen, diclofenac, ampiroxcam, and acetaminophen; synthetic adrenocortical hormone agents, such as dexamethasone palmitate, fluorometholone, betamethasone, and beclomethasone propionate; antimicrobial drugs, such as norfloxacin and levofloxacin; drugs for circulatory organ, such as tocopherol nicotinate; cerebral protective drugs, such as edaravone; drugs for liver disease, such as glycyrrhizinic acid-based compounds, for example, monoammonium glycyrrhizinate; prostaglandin compounds (compounds having a prostanoic acid backbone), such as prostaglandin, E₁, prostaglandin E₂, prostaglandin prostaglandin I₂, and various derivatives thereof, for example, alkyl esters (methyl ester, ethyl ester, propyl ester, butyl ester, and the like); vitamin E drugs, such as tocopherol acetate; contrast agents, such as iodinated poppy seed oil fatty acid ethyl ester; antiviral drugs, such as vidarabine, aciclovir, and adefovir pivoxil; antineoplastic drugs, such as mitomycin, irinotecan, etoposide, paclitaxel, docetaxel, cabazitaxel, ubenimex, carboplatin, and cisplatin.

In the present invention, examples of the oils or fats include known oils or fats that can be used as an oil or fat, such as vegetable oils, for example, soybean oil, corn oil, coconut oil, safflower oil, perilla oil, olive oil, castor oil, and cotton seed oil, animal oils, for example, lanolin, egg yolk oils, fish oils, mineral oils, for example, liquid paraffin, medium-chain triglycerides, chemosynthesis triglycerides, and gelated hydrocarbons. One of the oils or fats may be used alone or two or more thereof may be used in combination.

In the present invention, examples of the emulsifiers include lecithin (for example, egg yolk lecithin, soybean lecithin, hydrogenated egg yolk lecithin, hydrogenated soybean lecithin), polysorbate PEG-hydrogenated castor oil, polyoxyetle castor oil, and polyoxyethylene hydrogenated castor oil. One of the emulsifiers may be used alone or two or more thereof may be used in combination. Among them, lecithin is suitably used because of its high safety, in spite of its low emulsifying ability and its sometimes low handleability due to the high viscosity in high concentrations (lecithin may be used with another substance that can be used as an emulsifier in mixture, but in such a case, the proportion of lecithin in the emulsifiers is desirably 50% by weight or more, more desirably 55% by weight or more, and further desirably 60% by weight or more). Incidentally, in the case where the slightly water soluble drug is a prostaglandin compound, an emulsifier having a content of phosphatidylethanolamine of 2% by weight or less is desirably used (since phosphatidylethanolamine may negatively affect the stability of prostaglandin compounds). For example, PC-98N manufactured by Kewpie Corporation can be suitably used as a purified egg yolk lecithin obtained after a treatment of removing phosphatidylethanolamine (having a content of phosphatidylcholine of 98% by weight or more and a content of phosphatidylethanolamine of 1% by weight or less).

In the present invention, examples of the polyhydric alcohols used as a water soluble carrier include glycerol, diglycerol, polyglycerol, propylene glycol, diethylene triethylene glycol, and polyethylene glycol. One of the water soluble carriers may be used alone or two or more thereof may be used in combination. Among them, glycerol, propylene glycol, and polyethylene glycol are desirable in that they are water soluble and that they do not have fat solubility to such an extent that fat particles contained in a fat emulsion prepared by mixing the non-aqueous composition having a drug carried therein of the present invention with an aqueous medium are broken.

In the non-aqueous composition having a drug carried therein of the present invention, the reason why the content of the oil or fat is defined to 0.05 to 250 mg/g is as follow That is, with a content less than 0.05 mg/g, the amount of a slightly water soluble drug that can be supported by a fat emulsion prepared by mixing with an aqueous medium is small, whereas with a content larger than 250 mg/g, the amount of the oil or fat is so large that emulsification after mixing with an aqueous medium is difficult and thus it is difficult to prepare a fat emulsion. The content of the oil or fat is desirably 0.5 to 200 mg/g, and more desirably 1 to 150 mg/g. The reason why the weight ratio of the slightly water soluble drug to the oil or fat (slightly water soluble drug/(oil or fat)) is defined to 0.0001 to 50 (provided that the total content of the slightly water soluble drug and the oil or fat is at most 300 mg/g) is as follows. That is, with a ratio less than 0.0001, the oil or fat is excessive relative to the drug in a fat emulsion prepared by mixing with an aqueous medium so that some useless oil or fat will be administered to patients, whereas with a ratio larger than 50, the drug is excessive relative to the oil or fat so that the stability of the drug is impaired and the drug is likely to aggregate or precipitate. The weight ratio of the slightly water soluble drug to the oil or fat is desirably 0.001 to 20, and more desirably 0.01 to 10. The reason why the total content of the slightly water soluble drug and the oil or fat is defined to at most 300 mg/g is as follows. That is, with a total content larger than 300 mg/g, emulsification after mixing with an aqueous medium is difficult and thus it is difficult to prepare a fat emulsion. The total content of the slightly water soluble drug and the oil or fat is desirably 3 to 250 mg/g, and more desirably 5 to 200 mg/g. The reason why the content of the emulsifier is defined to 20 to 500 mg/g is as follows. That is, with a content less than 20 mg/g, the amount of the oil or fat relative to that of the emulsifier is so large that emulsification after mixing with an aqueous medium is difficult and thus it is difficult to prepare a fat emulsion, whereas with a content larger than 500 mg/g, the viscosity of a mixed liquid with an aqueous medium is so increased that emulsification is difficult and thus it is difficult to prepare a fat emulsion (this is notable especially when lecithin is used). The content of the emulsifier is desirably 50 to 400 mg/g, and more desirably 100 to 300 mg/g. When a weight ratio of the emulsifier to the oil or fat (emulsifier/(oil or fat)) is 1 to 300, emulsification after mixing with an aqueous medium is easy and thus it is easy to prepare a fat emulsion. Incidentally, the content of the slightly water soluble drug may be, for example, 0.01 to 50 mg/g. A larger amount of a drug is supported by a fat emulsion prepared by mixing with an aqueous medium, through dissolution in the oil or fat in cases of liposoluble drugs, or through coexistence with the emulsifier at an interface between water and the oil or fat in cases of non liposoluble drugs.

Incidentally, lactic acid, polyvinyl alcohol, polyvinyl pyrrolidone, methyl cellulose, chondroitin sulfate or a salt thereof (such as sodium salt), hyaluronic acid or s salt thereof (such as sodium salt), or glycyrrhizinic acid or s salt thereof (such as sodium salt or ammonium salt) may further be used as a component of the non-aqueous composition having a drug carried therein of the present invention in order to enhance the drug solubility to enhance the stability of the emulsion or the drug, or to isotonize the emulsion, for example, in the fat emulsion prepared by mixing with an aqueous medium. Such a component is desirably used at a content of 0.02 to 300 mg/mL in a prepared fat emulsion, and more desirably 0.2 to 100 mg/mL. With a content less than 0.0.2 mg/mL, the effect is hardly exhibited, whereas with a content larger than 300 mg/mL, the viscosity of a mixed liquid with an aqueous medium is so increased that emulsification is difficult and thus it is difficult to prepare a fat emulsion or the emulsion is unstable due to acidification thereof.

In addition, higher fatty acids having, for example, 14 to 24 carbon atoms (which may be in a salt form, the same applies hereinafter), such as oleic acid, stearic acid, linoleic acid, linolenic acid, palmitic acid, palmitoleic acid, and myristic acid, may further be used as a component of the non-aqueous composition having a drug carried therein of the present invention in order to stabilize the fat emulsion prepared by mixing with an aqueous medium. Such a higher fatty acid is desirably used at a content of 0.001 to 10 mg/mL in a prepared fat emulsion, and more desirably 0.01 to 5 mg/mL. With a content less than 0.001 mg/mL, the effect is hardly exhibited, whereas with a content larger than 10 mg/mL, there is a risk to degrade the drug. Incidentally, in the case where the slightly water soluble drug is a prostaglandin compound, such a higher fatty acid is desirably not used (since it may negatively affect the stability of prostaglandin compounds).

Higher fatty acids also have an effect of promoting an emulsifying action of an emulsifier (this effect is valuable when lecithin which has low emulsifying ability is used as the emulsifier). When a higher fatty acid is used as a component of the non-aqueous composition having a drug carried therein of the present invention in expectation of the effect, the higher fatty acid is desirably used at such a content that the weight ratio of the higher fatty acid to the emulsifier (higher fatty acid/emulsifier) is 0.1 to 1. When used at such a content, a higher fatty acid also has an effect of stabilizing a fat emulsion prepared.

In addition, middle fatty acids having, for example, 8 to 12 carbon atoms (which may be in a salt form), such as caprylic acid and capric acid, also have an effect of promoting an emulsifying action of an emulsifier as with higher fatty acids. When a middle fatty acid is used as a component of the non-aqueous composition having a drug carried therein of the present invention in expectation of the effect, the middle fatty acid is desirably used at such a content that the weight ratio of the middle fatty acid to the emulsifier (middle fatty acid/emulsifier) is 0.1 to 1. A higher fatty acid and a middle fatty acid may be mixed and used together. In this case, both are mixed at a desired ratio and the obtained fatty acid mixture is used at such a content that the weight ratio of the fatty acid mixture to the emulsifier (fatty acid mixture/emulsifier) is 0.1 to 1.

In addition, when a saccharide is further used as a component of the non-aqueous composition having a drug carried therein of the present invention, appearance of precipitated floaters which may appear in some cases in a fat emulsion prepared by mixing with an aqueous medium can be effectively suppressed. Suitable examples of saccharides include monosaccharides, such as inositol, glucose, sorbitol, fructose, and mannitol, disaccharides, such as trehalose, lactose, sucrose, and maltose, xylitol, dextrin, cyclodextrin, and dextran. Such a saccharide is desirably used at a content of 10 to 600 mg/mL in a prepared fat emulsion.

In addition, a pH modifier (such as citric acid) or an osmotic pressure modifier known per se may further be used as a component of the non-aqueous composition having a drug carried therein of the present invention in order to modify pH (for example, 4 to 8) or to modify osmotic pressure of a fat emulsion prepared by mixing with an aqueous medium. Incidentally, a preservative, an antioxidant, or the like may of course be used as a component as required. Also, the non-aqueous composition having a drug carried therein of the present invention is not discouraged from containing a water soluble drug as a component.

The non-aqueous composition having a drug carried therein of the present invention can be produced simply by dissolving an oil or fat, a drug, and an emulsifier in a polyhydric alcohol as a water soluble carrier at a content of the oil or fat, a weight ratio of the drug to the oil or fat, a total content of the drug and the oil or fat, and a content of the emulsifier which are set within the above numeral ranges. Since water is not used for producing the non-aqueous composition having a drug carried therein of the present invention, the composition contains no water (provided that moisture inevitably mixed therein can be present). The dissolution of the components in a polyhydric alcohol can be achieved, for example, with a mixing and stirring device. The non-aqueous composition having a drug carried therein of the present invention can be subjected to high pressure steam sterilization high pressure steam sterilization may be performed at common conditions (for example, at 120 to 122° C. for 10 to 15 minutes). In addition, the non-aqueous composition having a drug carried therein of the present invention is liquid, and thus can be subjected to filtration sterilization.

The non-aqueous composition having a drug carried therein of the present invention produced as described above is excellent in storage stability, and thus can be stored at room temperature (provided that this does not apply to the case where the drug is very unstable). By being mixed with an aqueous medium, such as water for injection, a physiological saline, and a sugar infusion (such as a glucose infusion), the non-aqueous composition having a drug carried therein of the present invention can prepare a drug-containing fat emulsion suitably having an average particle size of fat particles of 300 nm or less, more suitably 200 nm or less, and further suitably 100 nm or less (the lower limit is, for example, 1 nm), and suitably having a turbidity of 1.0 or lower, more suitably 0.8 or lower, and further suitably 0.5 or lower. The mixing of the non-aqueous composition having a drug carried therein of the present invention with the aqueous medium may be achieved by manually shaking them, for example, for 10 seconds to 2 minutes in use. The amount of the aqueous medium mixed with the non-aqueous composition having a drug carried therein of the present invention may be appropriately determined based on, for example, the amount of the drug carried in the non-aqueous composition to be administered. However, the fact that the thus-prepared drug-containing fat emulsion has transparency makes it easy to visually check occurrence of deterioration or contamination, and change in formulation, and provides a sense of safety to a patient whom the fat emulsion is administered. As described above, the non-aqueous composition having a drug carried therein of the present invention can be used as a dissolved-in-use type pharmaceutical preparation by itself, or can also be blended with various pharmaceutical additives (such as formulation additive) to provide various types of pharmaceutical preparations, such as an oral agent and external preparation.

### EXAMPLES

Hereinafter, the present invention is explained in detail with reference to examples, but the present invention should not be construed to be limited to the following description.

### Example 1

### Non-Aqueous Composition having Prostaglandin E₁ Carried Therein (1)

In a 50 mL beaker, 900 μg of prostaglandin E₁, 900 mg of medium-chain triglyceride (ODO: manufactured by The Nissin OilliO Group, Ltd., the same applies hereinafter), 3.6 g purified egg yolk lecithin (PC-98N: manufactured by Kewpie Corporation, the same applies hereinafter), 3.6 g of polysorbate (polysorbate 80), and 30 g of propylene glycol were taken, and while heating with a water bath at 45° C., the mixture was subjected to a dissolution treatment with a mixing and stirring device under nitrogen gas flow for about 10 minutes to obtain a target non-aqueous composition having prostaglandin E₁ carried therein (colorless transparent viscous liquid). 200 mg of the viscous liquid was taken in a test tube, was diluted 10-fold with pure water, and then was manually shaken for 1 minute. Thus, a colorless lightly-turbid prostaglandin E₁-containing fat emulsion was able to be prepared (see Tables 1 and 2).

### Example 2

### Non-Aqueous Composition having Tocopherol Acetate Carried Therein

In a 50 mL beaker, 60 mg of tocopherol acetate, 300 mg of medium-chain triglyceride (ODO), 3 g of purified egg yolk lecithin (PL-100M: manufactured by Kewpie Corporation, the same applies hereinafter), 2.4 g of polysorbate (polysorbate 80), and 30 g of propylene glycol were taken, and while heating with a water bath at 60° C. the mixture was subjected to a dissolution treatment with a mixing and stirring device for about 30 minutes to obtain a target non-aqueous composition having tocopherol acetate carried therein (yellow transparent viscous liquid). 200 mg of the viscous liquid was taken in a test tube, was diluted 10-fold with pure water, and then was manually shaken for 1 minute. Thus, a yellow lightly-turbid tocopherol acetate-containing fat emulsion was able to be prepared (see Tables 1 and 2).

### Example 3

### Non-Aqueous Composition having Dexamethasone Palmitate Carried Therein (1)

In a 50 mL beaker, 30 mg of dexamethasone palmitate, 300 mg of purified soybean oil, 3 g of purified egg yolk lecithin (PL-100M), 6 g of polysorbate (polysorbate 80), and 30 g of propylene glycol were taken, and while heating with a water bath at 60° C., the mixture was subjected to a dissolution treatment with a mixing and stirring device for about 15 minutes to obtain a target non-aqueous composition having dexamethasone palmitate carried therein (pale yellow transparent viscous liquid). 200 mg of the viscous liquid was taken in a test tube, was diluted 10-fold with pure water, and then was manually, shaken for 1 minute. Thus, a colorless transparent dexamethasone palmitate-containing fat emulsion was able to be prepared (see Tables 1 and 2).

### Example 4

### Non-Aqueous Composition having Prostaglandin E₁ Carried Therein (2)

In a 50 mL beaker, 900 μg of prostaglandin E₁, 300 mg of medium-chain triglyceride (ODO), 3 g of purified egg yolk lecithin (PC-98N), 1.8 g polysorbate (polysorbate 80), and 30 g of propylene glycol were taken, and while heating with a water bath at 45° C., the mixture was subjected to a dissolution treatment with a mixing and stirring device under nitrogen gas flow for about 15 minutes to obtain a target non-aqueous composition having prostaglandin E₁ carried therein (colorless transparent viscous liquid). 200 mg of the viscous liquid was taken in a test tube, was diluted 10-fold with pure water, and then was manually shaken for 1 minute. Thus, a colorless slightly-turbid prostaglandin E₁-containing fat emulsion was able to be prepared (see Tables 1 and 2).

### Example 5

### Non-Aqueous Composition having Docetaxel Carried Therein (1)

In a 30 mL beaker, 100 mg of docetaxel, 150 mg of purified soybean oil, 3 g of purified egg yolk lecithin (PL-100M) 3 g of polysorbate (polysorbate 80), and 20 g of propylene glycol were taken, and while heating with a water bath at 60° C., the mixture was subjected to a dissolution treatment with a mixing and stirring device for about 15 minutes to obtain a target non-aqueous composition having docetaxel carried therein (pale yellow transparent viscous liquid). 200 mg of the viscous liquid was taken in a test tube, was diluted 10-fold with pure water, and then was manually shaken for 1 minute. Thus, a pale yellow lightly-turbid docetaxel-containing fat emulsion was able to be prepared (see Tables 1 and 2).

### Example 6

### Non-Aqueous Composition having Docetaxel Carried Therein (2)

In a 30 mL beaker, 100 mg of docetaxel, 150 mg of purified soybean oil, 3 g of purified egg yolk lecithin (PL-100M), 9 g polysorbate (polysorbate 80), and 20 g of propylene glycol were taken, and while heating with a water bath at 60° C., the mixture was subjected to a dissolution treatment with a mixing and stirring device for about 15 minutes to obtain a target non-aqueous composition having docetaxel carried therein (pale yellow transparent viscous liquid). 200 mg of the viscous liquid was taken in a test tube, was diluted 10-fold with pure water, and then was manually shaken for 1 minute. Thus, a colorless transparent docetaxel-containing fat emulsion was able to be prepared (see Tables 1 and 2).

### Example 7

### Non-Aqueous Composition having Cabazitaxel Carried Therein

In a 50 mL beaker, 30 mg of cabazitaxel, 300 mg of medium-chain triglyceride (ODO), 4.5 g of purified egg yolk lecithin (PL-100M), 3.6 g of polysorbate (polysorbate 80), and 30 g of propylene glycol were taken, and while heating with a water bath at 60° C., the mixture was subjected to a dissolution treatment with a mixing and stirring device for about 15 minutes to obtain a target non-aqueous composition having cabazitaxel carried therein (pale yellow transparent viscous liquid). 200 mg of the viscous liquid was taken in a test tube, was diluted 10-fold with pure water, and then was manually shaken for 1 minute. Thus, a yellow turbid cabazitaxel-containing fat emulsion was able to be prepared (see Tables 1 and 2).

### Example 8

### Non-Aqueous Composition having Docetaxel Carried Therein (3)

In a 30 mL beaker, 100 mg of docetaxel, 150 mg of purified soybean oil, 9 g of purified egg yolk lecithin (PL-100M), 6 g of polysorbate (polysorbate 80), and20 g of propylene glycol were taken, and while heating with a water bath at 60° C., the mixture was subjected to a dissolution treatment with a mixing and stirring device for about 15 minutes to obtain a target non-aqueous composition having docetaxel carried therein (yellow transparent viscous liquid). 200 mg of the viscous liquid. was taken in a test tube, was diluted 10-fold with pure water, and then was manually shaken for 1 minute. Thus, a yellow turbid docetaxel-containing fat emulsion was able to be prepared (see Tables 1 and 2).

### Example 9

### Non-Aqueous Composition having Prostaglandin E₁ Carried Therein (3)

In a 50 mL, beaker, 900 μg of prostaglandin E₁, 300 mg of medium-chain triglyceride (ODO), 0.9 of purified egg yolk lecithin (PL-100M), 2.7 g of purified egg yolk lecithin (PC-98N), and 9 g of propylene glycol were taken, and while heating with a water bath at 45° C., the mixture was subjected to a dissolution treatment with a mixing and stirring device under nitrogen gas flow for about 15 minutes. Further, 7 g of glycerol heated to 60° C. was added thereto, and the mixture was subjected to a dissolution treatment for about 5 minutes to obtain a target non-aqueous composition having prostaglandin E₁ carried therein (pale yellow transparent viscous liquid). 200 mg of the viscous liquid was taken in a test tube, was diluted 10-fold with pure water, and then was manually shaken for 1 minute. Thus, a yellow lightly-turbid prostaglandin E₁-containing fat emulsion was able to be prepared (see Tables 1 and. 2).

### Example 10

### Non-Aqueous Composition having Dexamethasone Palmitate Carried Therein (2)

In a 50 mL beaker, 30 mg of dexamethasone palmitate, 30 mg of medium-chain triglyceride (ODO), 2.7 g of purified egg yolk lecithin (Pt-100M), 0.9 g of purified egg yolk lecithin (PC-98N), 9 g of propylene glycol, and 21 g of polyethylene glycol 400 (Macrogol 400) were taken, and while heating with a water bath at 60° C., the mixture was subjected to a dissolution treatment with a mixing and stirring device for about 20 minutes to obtain a target non-aqueous composition having dexamethasone palmitate carried therein (yellow transparent viscous liquid). 200 mg of the viscous liquid was taken in a test tube, was diluted 10-fold with pure water, and then was manually shaken for 1 minute. Thus, a yellow lightly-turbid dexamethasone palmitate-containing fat emulsion was able to be prepared (see Tables 1 and 2).

### Example 11

### Non-Aqueous Composition having Dexamethasone Palmitate Carried Therein (3)

In a 50 mL beaker, 30 mg of dexamethasone palmitate, 30 mg of medium-chain triglyceride (ODO), 1.8 g of purified egg yolk lecithin (PL-100M) 1.8 g of purified egg yolk lecithin (PC-98N), 24 g of propylene glycol, and 6 g of glycerol were taken, and while heating with a water bath at 60° C., the mixture was subjected to a dissolution treatment with a mixing and stirring device for about 20 minutes to obtain a target non-aqueous composition having dexamethasone palmitate carried therein (pale yellow transparent viscous liquid). 200 mg of the viscous liquid was taken in a test tube, was diluted 10-fold with pure water, and then was manually shaken for 1 minute. Thus, a yell ow lightly-turbid dexamethasone palmitate-containing fat emulsion was able to be prepared (see Tables 1 and 2).

### Example 12

### Non-Aqueous Composition having Dexamethasone Palmitate Carried Therein (4)

In a 50 mL beaker, 30 mg of dexamethasone palmitate, 300 mg of purified soybean oil, 3 g of purified egg yolk lecithin (PL-100M), 6 g of polyoxyethylene hydrogenated castor oil (HCO-60: manufactured by Nikko Chemicals Co. , Ltd., the same applies hereinafter), and 30 g of propylene glycol were taken, and while heating with a water bath at 60° C., the mixture was subjected to a dissolution treatment with a mixing and stirring device for about 15 minutes to obtain a target non-aqueous composition having dexamethasone palmitate carried therein (pale yellow transparent viscous liquid). 200 mg of the viscous liquid was taken in a test tube, was diluted 10-fold with pure water, and then was manually shaken for 1 minute. Thus, a colorless transparent dexamethasone palmitate-containing fat emulsion was able to be prepared (see Tables 1 and 2).

### Example 13

### Non-Aqueous Composition containing Fat Particles having Docetaxel Carried Therein (4)

In a 50 mL beaker, 100 mg of docetaxel, 150 mg of purified soybean oil, 3 g of purified egg yolk lecithin (PL-100M), 9 g of polyoxyethylene castor oil (UNION C-35: manufactured by NOF CORPORATION), and 20 g of propylene glycol were taken, and while heating with a water bath at 60° C., the mixture was subjected to a dissolution treatment with an ultrasonic device for about 15 minutes to obtain a target non-aqueous composition having docetaxel carried therein (pale yellow transparent viscous liquid). 200 mg of the viscous liquid was taken in a test tube, was diluted 10-fold with pure water, and then was manually shaken for 1 minute. Thus, a colorless transparent docetaxel-containing fat emulsion was able to be prepared (see Tables 1 and 2).

### Example 14

### Non-Aqueous Composition Containing Fat Particles having Docetaxel Carried Therein

In a 50 mL beaker, 100 mg of docetaxel, 150 mg of purified soybean oil, 3 g of purified egg yolk lecithin (PL-100M), 9 g of polyoxyethylene hydrogenated castor oil (HCO-60), and 20 g of propylene glycol were taken, and while heating with a water bath at 60° C., the mixture was subjected to a dissolution treatment with an ultrasonic device for about 15 minutes to obtain a target non-aqueous composition having docetaxel carried therein (pale yellow transparent viscous liquid). 200 mg of the viscous liquid was taken in a test tube, was diluted 10-fold with pure water, and then was manually shaken for 1 minute. Thus, a pale yellow slightly-turbid docetaxel-containing fat emulsion was able to be prepared (see Tables 1 and 2).

### Example 15

### Non-Aqueous Composition Containing Fat Particles having Dexamethasone Palmitate Carried Therein (5)

In a 5 mL micro test tube, 360 mg of purified egg yolk lecithin (PL-100M) and 3.236 g of propylene glycol were taken, and the mixture was subjected to a dissolution treatment with an ultrasonic device for about 3 minutes (treated liquid A). In another 5 mL micro test tube, 4 mg of dexamethasone palmitate, 40 mg of medium-chain triglyceride (ODO), and 360 mg of polysorbate (polysorbate 80) were taken, and after heating with a hot bath at 80° C., the mixture was subjected to a dissolution treatment with an ultrasonic device for about 5 minutes (treated liquid B). The treated liquid B was added to the treated liquid A, and the mixture was subjected to a dissolution treatment with an ultrasonic device for about 3 minutes to obtain a target non-aqueous composition having dexamethasone palmitate carried therein (yellow transparent viscous liquid). 200 mg of the viscous liquid was taken in a test tube, was diluted 10-fold with pure water, and then was manually shaken for 1 minute. Thus, a yellow turbid dexamethasone palmitate-containing fat emulsion was able to be obtained (see Tables 1 and 2).

### Example 16

### Non-Aqueous Composition Containing Fat Particles having Dexamethasone Palmitate Carried Therein (6)

In a 5 mL micro test tube, 360 mg of purified egg yolk (PL-100M) and 1.876 g of propylene glycol were taken, and the mixture was subjected to a dissolution treatment with an ultrasonic device for about 3 minutes (treated liquid A). In another 5 mL micro test tube, 4 mg of dexamethasone palmitate and 40 mg of medium-chain triglyceride (ODO) were taken, and after heating with a hot bath at 80° C., the mixture was subjected to a dissolution treatment with an ultrasonic device for about 3 minutes (treated liquid B). In further another 5 mL micro test tube, 120 mg of polysorbate (polysorbate 80) and 1.6 g of glycerol were taken, and the mixture was subjected to a dissolution treatment with an ultrasonic device for about 2 minutes (treated liquid C). The treated liquid B was added to the treated liquid A, the mixture was subjected to a dissolution treatment with an ultrasonic device for about 3 minutes, and then the treated liquid C was added thereto. The mixture was further subjected to a dissolution treatment with an ultrasonic device for about 3 minutes to obtain a target non-aqueous composition having dexamethasone palmitate carried therein (yellow slightly-turbid viscous liquid) 200 mg of the viscous liquid was taken in a test tube, was diluted 10-fold with pure water, and then was manually shaken for 1 minute. Thus, a yellow turbid dexamethasone palmitate-containing fat emulsion was able to be obtained (see Tables 1 and 2).

### Example 17

### Non-Aqueous Composition Containing Fat Particles having Dexamethasone Palmitate Carried Therein (7)

In a 5 mL micro test tube, 3 mg of dexamethasone palmitate, 3 mg of purified soybean oil, 180 mg of purified egg yolk lecithin (PL-100M), 180 mg of purified egg yolk lecithin (PC-98N), and 2.634 g of propylene glycol were taken, and the mixture was subjected to a dissolution treatment with an ultrasonic device for about 3 minutes. Then, 150 mg of sorbitol was added thereto, and the mixture was further subjected to a dissolution treatment with an ultrasonic device for about 2 minutes to obtain a target non-aqueous composition having dexamethasone palmitate carried therein (pale yellow transparent viscous liquid). 300 mg of the viscous liquid was taken in a test tube, was diluted 10-fold with pure water, and then was manually shaken for 1 minute. Thus, a pale yellow turbid dexamethasone palmitate-containing fat emulsion was able to be obtained (see Tables 1 and 2).

### Example 18

### Non-Aqueous Composition Containing Fat Particles having Dexamethasone Palmitate Carried Therein (8)

In a 5 mL micro test tube, 30 mg of dexamethasone palmitate and 30 mg of medium-chain triglyceride (ODO) were taken, and after heating with a hot bath at 80° C., the mixture was subjected to a dissolution treatment with an ultrasonic device for about 3 minutes (treated liquid A). In another 5 mL micro test tube, 600 mg of purified egg yolk lecithin (PL-100M), 1.17 g of propylene glycol , and 1.17 g of polyethylene glycol 300 (Macrogol 300) were taken, and the mixture was subjected to a dissolution treatment with an ultrasonic device for about 2 minutes (treated liquid B). The treated liquid A was added to the treated liquid B, and the mixture was subjected to a dissolution treatment with an ultrasonic device for about 3 minutes to obtain a target non-aqueous composition having dexamethasone palmitate carried therein (yellow transparent viscous liquid). 200 mg of the viscous liquid was taken in a test tube, was diluted 30-fold with pure water, and then was manually shaken for 1 minute. Thus, a yellow turbid dexamethasone palmitate-containing fat emulsion was able to be obtained (see Tables 1 and 2).

### Example 19

### Non-Aqueous Composition Containing Fat Particles having Dexamethasone Palmitate Carried Therein (9)

In a 5 mL micro test tube, 25 mg of dexamethasone palmitate and 75 mg of medium-chain triglyceride (ODO) were taken, and after heating with a hot bath at 80° C., the mixture was subjected to a dissolution treatment with an ultrasonic device for about 3 minutes. Then, 500 mg of purified egg yolk lecithin (PL-100M), 500 mg of sodium oleate, and 2 g of propylene glycol were added thereto, and the mixture was further subjected to a dissolution treatment with an ultrasonic device for about 3 minutes to obtain a target non-aqueous composition having dexamethasone palmitate carried therein (yellow transparent viscous liquid). 200 mg of the viscous liquid was taken in a test tube, was diluted 30-fold with pure water, and then was manually shaken for 1 minute. Thus, a colorless transparent dexamethasone palmitate-containing fat emulsion was able to be obtained (see Tables 1 and 2)

### Example 20

### Non-Aqueous Composition Containing Fat Particles having Dexamethasone Palmitate Carried Therein (10)

In a 5 mL micro test tube, 25 mg of dexamethasone palmitate and 75 mg of medium-chain triglyceride (ODO) were taken, and after heating with a hot bath at 80° C., the mixture was subjected to a dissolution treatment with an ultrasonic device for about 3 minutes. Then, 500 mg of purified egg yolk lecithin (PC-98N), 500 mg of sodium oleate, and 2 g of propylene glycol were added thereto, and the mixture was further subjected to a dissolution treatment with an ultrasonic device for about 3 minutes to obtain a target non-aqueous composition having dexamethasone palmitate carried therein (slightly-yellow transparent viscous liquid). 200 mg of the viscous liquid was taken in a test tube, was diluted 30-fold with pure water, and then was manually shaken for 1 minute. Thus, a colorless transparent dexamethasone palmitate-containing fat emulsion was able to obtained (see Tables 1 and 2).

### Example 21

### Non-Aqueous Composition Containing Fat Particles having Dexamethasone Palmitate Carried Therein (11)

In a 5 mL micro test tube, 25 mg of dexamethasone palmitate and 75 mg of medium-chain triglyceride (ODO) were taken, and after heating with a hot bath at 80° C., the mixture was subjected to a dissolution treatment with an ultrasonic device for about 3 minutes . Then, 250 mg of purified egg yolk lecithin (PC-98N), 250 mg of sodium oleate, and 1.5 g of propylene glycol were added thereto, and the mixture was further subjected to a dissolution treatment with an ultrasonic device for about 3 minutes to obtain a target non-aqueous composition having dexamethasone palmitate carried therein (slightly-yellow transparent viscous liquid). 200 mg of the viscous liquid was taken in a test tube, was diluted 30-fold with pure water, and then was manually shaken for 1 minute. Thus, a colorless transparent dexamethasone palmitate-containing fat emulsion was able to be obtained (see Tables 1 and 2).

### Example 22

### Non-Aqueous Composition Containing Fat Particles having Cyclosporin Carried Therein (1)

In a 5 mL micro test tube, 150 me of cyclosporin, 450 mg of medium-chain triglyceride (ODO), 600 mg of purified egg yolk lecithin (Pt-100M), 300 mg of sodium oleate, and 1.5 g of propylene glycol were taken, and the mixture was subjected to a dissolution treatment with an ultrasonic device for about 5 minutes to obtain a target non-aqueous composition having cyclosporin carried therein (yellow transparent viscous liquid). 200 mg of the viscous liquid was taken in a test tube, was diluted 30-fold with pure water, and then was manually shaken for 1 minute. Thus, a yellow lightly-turbid cyclosporin-containing fat emulsion was able to be obtained (see Tables 1 and 2).

### Example 23

### Non-Aqueous Composition Containing Fat Particles having Paclitaxel Carried Therein (1)

In a 5 mL micro test tube, 50 mg of paclitaxel, 250 mg of purified soybean oil, 500 mg of purified egg yolk lecithin (PL-100M), 500 mg of sodium oleate, and 1.7 g of propylene glycol were taken, and the mixture was subjected to a dissolution treatment with an ultrasonic device for about 5 minutes to obtain a target non-aqueous composition having paclitaxel carried therein (yellow transparent viscous liquid). 200 mg of the viscous liquid was taken in a test tube, was diluted 30-fold with pure water, and then was manually shaken for 1 minute. Thus, a colorless transparent paclitaxel-containing fat emulsion was able to be obtained (see Tables 1 and 2).

### Example 24

### Non-Aqueous Composition Containing Fat Particles having Ciclosporin Carried Therein (2)

In a 5 mL micro test tube, 75 mg of cyclosporin, 300 mg of medium-chain tritriglyceride (ODO), 600 mg of purified egg yolk lecithin (PC-98N), 150 mg of sodium oleate, and 1.875 g of propylene glycol were taken, and the mixture was subjected to a dissolution treatment with an ultrasonic device for about 3 minutes to obtain a target non-aqueous composition having cyclosporin carried therein (slightly-yellow transparent viscous liquid). 200 mg of the viscous liquid was taken in a test tube, was diluted 30-fold with pure water, and then was manually shaken for 1 minute. Thus, a colorless transparent cyclosporin-containing fat emulsion was able to be obtained (see Tables 1 and 2).

### Example 25

### Non-Aqueous Composition Containing Fat Particles having Dexamethasone Palmitate Carried Therein (12)

In a 5 mL micro test tube, 3.75 mg of dexamethasone palmitate, 3.75 mg of medium-chain triglyceride (ODO), 225 mg of purified egg yolk lecithin (PL-100M), 225 mg of purified egg yolk lecithin (PC-98N), and 3 g of propylene glycol were taken, and the mixture was subjected to a dissolution treatment with an ultrasonic device for about 5 minutes. Then, 150 mg of sorbitol was added thereto, and the mixture was further subjected to a dissolution treatment with an ultrasonic device for about 2 minutes to obtain a target non-aqueous composition having dexamethasone palmitate carried therein (pale yellow transparent viscous liquid). 200 mg of the viscous liquid was taken in a test tube, was diluted 30-fold with pure water, and then was manually shaken for 1 minute. Thus, a pale yellow lightly-turbid dexamethasone palmitate-containing fat emulsion was able to be obtained (see Tables 1 and 2).

### Example 26

### Non-Aqueous Composition Containing Fat Particles having Dexamethasone Palmitate Carried Therein (13)

In a 5 mL micro test tube, 50 mg of dexamethasone palmitate and 100 mg of medium-chain triglyceride (ODO) were taken, and after heating with a hot bath at 80° C., the mixture was subjected to a dissolution treatment with an ultrasonic device for about 3 minutes Then, 500 mg of purified egg yolk lecithin (PL-100M) 250 mg of sodium oleate, 250 mg of sodium caprylate, and 1.85 g of propylene glycol were added thereto, and the mixture was further subjected to a dissolution treatment with an ultrasonic device for about 3 minutes to obtain a target non-aqueous composition having dexamethasone palmitate carried therein (pale yellow transparent viscous liquid). 200 mg of the viscous liquid was taken in a test tube, was diluted 30-fold with pure water, and then was manually shaken for 1 minute. Thus, a colorless transparent dexamethasone palmitate-containing fat emulsion was able to be obtained (see Tables 1 and 2).

TABLE 1

| | Composition of non-aqueous composition | | | | |
|---|---|---|---|---|---|
| | | Water solubility of drug | Concentration | Concentration | Concentration |
| | | (Expression in Japanese | of drug | of oil or fat | of emulsifier |
| | Drug | Pharmacopoeia) | (mg/g) | (mg/g) | (mg/g) |
| Ex. 1 | Prostaglandin E₁ | Practically insoluble | 0.024 | 23.6 | 189 |
| Ex. 2 | Tocopherol acetate | Practically insoluble | 1.68 | 8.4 | 151 |
| Ex. 3 | Dexamethasone palmitate | Practically insoluble | 0.76 | 7.6 | 229 |
| Ex. 4 | Prostaglandin E₁ | Practically insoluble | 0.026 | 8.5 | 137 |
| Ex. 5 | Docetaxel | Practically insoluble | 3.81 | 5.7 | 229 |
| Ex. 6 | Docetaxel | Practically insoluble | 3.10 | 4.7 | 372 |
| Ex. 7 | Cabazitaxel | Practically insoluble | 0.78 | 7.8 | 211 |
| Ex. 8 | Docetaxel | Practically insoluble | 2.84 | 4.3 | 426 |
| Ex. 9 | Prostaglandin E₁ | Practically insoluble | 0.045 | 15.1 | 181 |
| Ex. 10 | Dexamethasone palmitate | Practically insoluble | 0.89 | 0.9 | 107 |
| Ex. 11 | Dexamethasone palmitate | Practically insoluble | 0.89 | 0.9 | 107 |
| Ex. 12 | Dexamethasone palmitate | Practically insoluble | 0.76 | 7.6 | 229 |
| Ex. 13 | Docetaxel | Practically insoluble | 3.10 | 4.7 | 372 |
| Ex. 14 | Docetaxel | Practically insoluble | 3.10 | 4.7 | 372 |
| Ex. 15 | Dexamethasone palmitate | Practically insoluble | 1.00 | 10.0 | 180 |
| Ex. 16 | Dexamethasone palmitate | Practically insoluble | 1.00 | 10.0 | 120 |
| Ex. 17 | Dexamethasone palmitate | Practically insoluble | 0.95 | 1.0 | 114 |
| Ex. 18 | Dexamethasone palmitate | Practically insoluble | 10.0 | 10.0 | 200 |
| Ex. 19 | Dexamethasone palmitate | Practically insoluble | 8.06 | 24.2 | 161 |
| Ex. 20 | Dexamethasone palmitate | Practically insoluble | 8.06 | 24.2 | 161 |
| Ex. 21 | Dexamethasone palmitate | Practically insoluble | 11.9 | 35.7 | 119 |
| Ex. 22 | Ciclosporin | Practically insoluble | 50.0 | 150.0 | 200 |
| Ex. 23 | Paclitaxel | Practically insoluble | 16.7 | 83.3 | 167 |
| Ex. 24 | Ciclosporin | Practically insoluble | 25.0 | 100.0 | 200 |
| Ex. 25 | Dexamethasone palmitate | Practically insoluble | 1.04 | 1.0 | 125 |
| Ex. 26 | Dexamethasone palmitate | Practically insoluble | 16.7 | 33.3 | 167 |

TABLE 2

| | Non-aqueous composition | | | Drug containing fat emulsion | | |
|---|---|---|---|---|---|---|
| | | Water soluble | | | | Average particle size |
| | Drug | carrier | Properties | Appearance | Turbidity | (nm) |
| Ex. 1 | Prostaglandin E₁ | Propylene glycol | Colorless transparent | Colorless | 0.474 | 173.4 |
| | | | viscous liquid | lightly-turbid | | |
| Ex. 2 | Tocopherol | Propylene glycol | Yellow transparent | Yellow | 0.466 | 179.2 |
| | acetate | | viscous liquid | lightly-turbid | | |
| Ex. 3 | Dexamethasone | Propylene glycol | Pale yellow transparent | Colorless | 0.000 | 67.9 |
| | palmitate | | viscous liquid | transparent | | |
| Ex. 4 | Prostaglandin E₁ | Propylene glycol | Colorless transparent | Colorless | 0.343 | 166.6 |
| | | | viscous liquid | slightly-turbid | | |
| Ex. 5 | Docetaxel | Propylene glycol | Pale yellow transparent | Pale yellow | 0.367 | 177.5 |
| | | | viscous liquid | lightly-turbid | | |
| Ex. 6 | Docetaxel | Propylene glycol | Pale yellow transparent | Colorless | 0.000 | 24.9 |
| | | | viscous liquid | transparent | | |
| Ex. 7 | Cabazitaxel | Propylene glycol | Pale yellow transparent | Yellow | 0.828 | 256.9 |
| | | | viscous liquid | turbid | | |
| Ex 8 | Docetaxel | Propylene glycol | Yellow transparent | Yellow | 1.046 | 249.4 |
| | | | viscous liquid | turbid | | |
| Ex. 9 | Prostaglandin E₁ | Propylene glycol | Pale yellow transparent | Yellow | 0.769 | 288.3 |
| | | Glycerol | viscous liquid | lightly-turbid | | |
| Ex. 10 | Dexamethasone | Propylene glycol | Yellow transparent | Yellow | 0.724 | 218.0 |
| | palmitate | Polyethylene glycol | viscous liquid | lightly-turbid | | |
| Ex. 11 | Dexamethasone | Propylene glycol | Pale yellow transparent | Yellow | 1.130 | 284.4 |
| | palmitate | Glycerol | viscous liquid | lightly-turbid | | |
| Ex. 12 | Dexamethasone | Propylene glycol | Pale yellow transparent | Colorless | 0.022 | 69.6 |
| | palmitate | | viscous liquid | transparent | | |
| Ex. 13 | Docetaxel | Propylene glycol | Pale yellow transparent | Colorless | 0.034 | 67.1 |
| | | | viscous liquid | transparent | | |
| Ex. 14 | Docetaxel | Propylene glycol | Pale yellow transparent | Pale yellow | 0.108 | 164.1 |
| | | | viscous liquid | slightly-turbid | | |
| Ex. 15 | Dexamethasone | Propylene glycol | Yellow transparent | Yellow | 0.429 | 198.1 |
| | palmitate | | viscous liquid | turbid | | |
| Ex. 16 | Dexamethasone | Propylene glycol | Yellow slightly-turbid | Yellow | 0.435 | 220.1 |
| | palmitate | Glycerol | viscous liquid | turbid | | |
| Ex. 17 | Dexamethasone | Propylene glycol | Pale yellow transparent | Pale yellow | 0.651 | 605.5 |
| | palmitate | | viscous liquid | turbid | | |
| Ex. 18 | Dexamethasone | Propylene glycol | Yellow transparent | Yellow | 0.652 | 724.7 |
| | palmitate | Polyethylene glycol | viscous liquid | turbid | | |
| Ex. 19 | Dexamethasone | Propylene glycol | Yellow transparent | Colorless | 0.049 | 121.0 |
| | palmitate | | viscous liquid | transparent | | |
| Ex. 20 | Dexamethasone | Propylene glycol | Slightly-yellow transparent | Colorless | 0.028 | 74.9 |
| | palmitate | | viscous liquid | transparent | | |
| Ex. 21 | Dexamethasone | Propylene glycol | Slightly-yellow transparent | Colorless | 0.011 | 17.4 |
| | palmitate | | viscous liquid | transparent | | |
| Ex. 22 | Ciclosporin | Propylene glycol | Yellow transparent | Yellow | 0.486 | 187.0 |
| | | | viscous liquid | lightly-turbid | | |
| Ex. 23 | Paclitaxel | Propylene glycol | Yellow transparent | Colorless | 0.002 | 8.4 |
| | | | viscous liquid | transparent | | |
| Ex. 24 | Ciclosporin | Propylene glycol | Slightly-yellow transparent | Colorless | 0.016 | 81.4 |
| | | | viscous liquid | transparent | | |
| Ex. 25 | Dexamethasone | Propylene glycol | Pale yellow transparent | Pale yellow | 0.374 | 616.7 |
| | palmitate | | viscous liquid | lightly-turbid | | |
| Ex. 26 | Dexamethasone | Propylene glycol | Pale yellow transparent | Colorless | 0.007 | 54.8 |
| | palmitate | | viscous liquid | transparent | | |

Incidentally, the turbidity was measured using an ultraviolet spectrophotometer (UV1800: manufactured by Shimadzu Corporation) at a wavelength of λ=620 nm with a sample placed in a measurement cell having a width of 1 cm (water was used as blank). A range of transparent to translucent at which a sample looks transparent, and occurrence of deterioration, such as aggregation or sedimentation, or contamination, or change in formulation can be easily visually checked, is Abs (absorbance) of 0.5 or lower. The average particle size was measured using a particle size analyzer (Zetasizer Nano ZS: manufactured by Malvern Palalytical Ltd.) which is based on the photon correlation method.

### Preparation Example 1

### Prostaglandin E₁-Containing Liquid for Injection

The non-aqueous composition having prostaglandin E₁ carried therein (colorless transparent viscous liquid) obtained in Example 1 itself was used as a prostaglandin E₁-containing liquid for injection.

### Preparation Example 2

### Prostaglandin E₁-Containing Ointment

To 11.9 g of the non-aqueous composition having prostaglandin E₁ carried therein (colorless transparent viscous liquid) obtained in Example 4, 88.1 g of Macrogol ointment melted with heat at 60° C. was slowly added with stirring The mixture was uniformly mixed and was then cooled and solidified to thereby obtain a prostaglandin E₁-containing ointment.

### Preparation Example 3

### Prostaglandin E₁-Containing Gel

11.9 g of the non-aqueous composition having prostaglandin E₁ carried therein (pale yellow transparent viscous liquid) obtained in Example 9 was mixed with 25 mL of water for injection, then 75 mL of a 4.5% carmellose sodium solution was added thereto, and the mixture was fully kneaded to thereby obtain a prostaglandin E₁-containing gel.

### INDUSTRIAL APPLICABILITY

The present invention has an industrial applicability in the point of capability of providing a non-aqueous composition having a drug carried therein, the composition being capable of preparing a drug-containing fat emulsion that is usable as an injection, an eye drop, a nasal drop, an inhalant, or the like by being mixed with an aqueous medium in use without producing a drug-containing fat emulsion in advance, and a method for producing the non-aqueous composition.

1. A non-aqueous composition having a drug carried therein, characterized by comprising an oil or fat, a slightly water soluble drug, and an emulsifier which are dissolved in a polyhydric alcohol as a water soluble carrier at a content of the oil or fat of 0.05 to 250 mg/g, a weight ratio of the slightly water soluble drug to the oil or fat (slightly water soluble drug/(oil or fat)) of 0.0001 to 50 (provided that the total content of the slightly water soluble drug and the or fat is at most 300 mg/g), and a content of the emulsifier of 20 to 500 mg/g.

2. The non-aqueous composition according to claim 1, characterized in that the polyhydric alcohol is at least one selected from glycerol, propylene glycol, and polyethylene glycol.

3. A method for producing a nonaqueous composition having a drug carried therein, characterized by comprising dissolving an oil or fat, a slightly water soluble drug, and an emulsifier in a polyhydric alcohol as a water soluble carrier at a content of the oil or fat of 0.05 to 250 mg/g, a weight ratio of the slightly water soluble drug to the oil or fat (slightly water soluble drug/(oil or fat)) of 0.0001 to 50 (provided that the total content of the slightly water soluble drug and the oil or fat is at most 300 mg/g), a content of the emulsifier of 20 to 500 mg/g.

4. A pharmaceutical preparation, characterized by comprising the non-aqueous composition having a drug carried therein according to claim 1 alone or together with another component blended therein.
